(19) United States
(12) Patent Application Publication
(10) Pub. No.: US 2016/0183556 A1
(43) Pub. Date: Jun. 30, 2016

(54) METHOD FOR PRODUCING FEED ADDITIVE CONTAINING SURFACTIN

(71) Applicant: SAFT biotechnology com. LTD, Nanjing (CN)

(72) Inventors: Jenn-Kan LU, New Taipei City (TW); Yi-Peng LIN, Taipei City (TW); Hsin-Mei WANG, Pingtung City (TW)

(21) Appl. No.: 14/966,698

(22) Filed: Dec. 11, 2015

(30) Foreign Application Priority Data
Dec. 12, 2014 (TW) 103143406

(51) Int. Cl. A23K 3/03; C12N 1/20; A61K 38/12; C12R 1/125; A61K 35/742

(52) U.S. Cl. CPC A23K 1/007; C12R 1/125; C12N 1/20; A61K 35/742; A61K 38/12; A23K 1/17; A23K 1/14

(57) ABSTRACT

A method for preparation of a feed additive containing surfactin, which uses a high-yield mutant strain, Bacillus subtilis T, and soybeans as the substrate in semi-solid state fermentation and after drying and grinding of the fermented soybeans into soybean powder to give a feed additive containing surfactin. Each kilogram of the fermented soybean powder contains about 6-7 g of surfactin. Feeding grouper with the feed additive containing surfactin can promote growth rate, increase expression of immune genes (e.g. AMP, Mx and IFN-induced protein), and lower mortality rate of virus-infected fish, indicating the feed containing surfactin has the effects of growth promotion, immunity enhancement and provides resistance to pathogen infection.

### BACKGROUND OF THE INVENTION

1. Field of the Invention

The present invention belongs to the field of aquaculture technology and biotechnology in the field of agriculture, involving a method of producing surfactin, a lipopeptide produced by Bacillus subtilis and its application in aquafeeds.

2. Description of the Prior Art

The secondary metabolites produced by Bacillus subtilis include β-lactams, aminoglycoside, polyketides and small polypeptide, among which lipopeptides and lipoproteins have attracted much attention because of their antibacterial effects and thus are also called Antimicrobial Peptide (AMP).

All AMPS discovered so far can be divided into three families. First, Fengycin which exists as a ring of a peptide chain containing 10 amino acids with 14 to 18 fatty acid side chains attached to its N-terminus has superior anti-fungal activity but shows no apparent effects on yeasts and bacteria (Schneider et al., 1999). The second family is Iturin which comprises a loop of seven α-amino acids coupled with β-amino acids and alkyl chains and the peptide chain is connected with 14-17 branched fatty acid chains, having a molecular weight of about 1044˜1081 kD. It provides anti-fungal and anti-bacterial effects, is biodegradable, and has high surface activity and low toxicity (Bonmatin, Laprevote, & Peypoux, 2003). The third family is surfactin, discussed in this invention, which contains 7 rings of amino acid chains with 13 to 16 side chains of fatty acids. Production of surfactin by Bacillus subtilis is usually accompanied by a subtilin. Co-existence of the two antimicrobial peptides will increase hemolysis of red blood cells due to the high affinity of the mixed micelles to cells and therefore consequently increase the anti-fungal activity (Feignier, Besson, & Michel, 1995; R Maget-Dana, Thimon, Peypoux, & Ptak, 1992; Sandrin, Peypoux, & Michel, 1990).

In 1968, Arima et al. found a white needle-like crystal in the culture medium of Bacillus subtilis and named it surfactin because of its surface activity (Kakinuma, Hori, Isono, Tamura, & Arima, 1969). From previous studies, the surface tension of water can be reduced from 72 mN/m to 27 mN/m by adjusting the concentration of surfactin to 20 μM and surfactin is the best biosurfactant identified so far (Arima, Kakinuma, & Tamura, 1968). Surfactin is a ring of lipopeptides containing contains 7 rings of amino acid chains with 13 to 16 side chains of fatty acids (Kakinuma, Sugino, Isono, Tamura & Arima, 1969). In solutions, the carbon chain of a fatty acid is hydrophobic. On the other hand, the rings of amino acids are the hydrophilic ends, aspartic acid and glutamic acid are both negatively charged, and the two negatively charged amino acids will form a clamp which makes surfactin a structure of negatively charged saddle and belongs to the family of anionic biosurfactants (Tsan, Volpon, Besson & Lancelin, 2007). According to the sequences of amino acids they contain, surfactin can be categorized into four types: A, B, C and D. The major difference is that the number of fatty acid chains attached to the rings of amino acids varies (Shaligram & Singhal, 2010; Singh & Cameotra, 2004).

Because of the non-specific bioactivity of surfactin, it can destroy the cell membrane of bacteria by reducing surface tension without producing drug-resistance. It is therefore considered to have antimicrobial potential and categorized as the AMP family (Cho, Lee, Cha, Kim & Shin, 2003). Past research has indicated that surfactin can inhibit fungal, mycoplasma, Gram-negative bacteria and Gram-positive bacteria growth (P. Das, Mukherjee & Sen, 2008; Singh & Cameotra, 2004). Heiko et al. reported that surfactin has the antimicrobial effect at the concentration of 12˜50 μg/ml (Heerklotz & Seelig, 2001). Lipopeptide biosurfactants produced by Bacillus circulans in the ocean can inhibit the growth of certain multidrug-resistant strain (MDR) and methicillin-resistant Staphylococcus aureus (MRSA) (Pan et al., 2007). Moreover, surfactin also has anti-fungal activity and can restore morphological features of cells contaminated by mycoplasma and produces no harm to metabolism and proliferation of the cell (Vollenbroich, Pauli, Ozel, & Vater, 1997). Surfactin was found to cause perforation of mycoplasma membrane by electron microscopy and will lead to death of mycoplasma due to imbalance of outer and inner osmotic pressure (Regine Maget-Dana & Ptak, 1995).

Studies have suggested the anti-viral mechanism of surfactin is caused by structural breakdown of viral envelope and protein coat due to physical and chemical interactions between the lipid envelope of a virus and surfactin as well as inhibition of replication of viral nucleic acid (Vollenbroich, Ozel, Vater, Kamp, & Pauli, 1997). Past studies have pointed out that surfactin is capable of inhibiting the growth of several viruses, including Human Immunodeficiency Virus (HIV), Suid Herpes virus type 1 (SHV-1), Herpes Simplex Virus 1 (HSV-2), Vesicular Stomatitis Virus (VSV), Simian Immunodeficiency Virus (SIV), Feline Calicivirus (FCV), Murine Encephalomyocarditis Virus (MECV), Pseudorabies Virus (PRV), Porcine Parvovirus (PPV), Newcastle Disease Virus (NDV) and Infectious Bursal Disease Virus (IBDV) (Huang et al., 2006; Itokawa et al., 1994; Vollenbroich, Özel, et al., 1997).

The antimicrobial mechanism of antibiotics is binding of antibiotics to the receptors of specific sites on pathogens so that the normal structure of pathogens is destroyed or certain biosynthesis processes blocked, resulting in bacteriostatic or bactericidal effect. Some studies have focused on exploring the functions of surfactin from the angle of antimicrobial peptides. Surfactin and AMP are both amphiphilic molecules. Surfactin is a ring structure, whereas AMPs can be divided into four classes based on their structures: α-helix, β-sheet, β-turn loop, and boat-shaped. The mechanism of interactions between AMPs and membrane is peptide-lipid interaction which is further categorized into four models: aggregate model, toroidal pore model, barrel-Stave model, and carpet model, and most of these models cause death of bacteria through the interactions between lipopeptides by imbalanced inner and outer osmotic pressure. No definite mechanism of the interaction between surfactin and cell membrane has been confirmed thus far. Nonetheless, a number of hypotheses have been proposed:

1. Detergent-like. This mechanism refers to insertion of a portion of the peptide chain into the middle layer of the cell membrane which causes stronger destabilization of the cell membrane and subsequently results in leak and partitioning of the membrane (Lohner and Epand, 1997; Heeklotz et al., 2004).

2. Formation of vesicles. This mechanism can be divided into three parts: (1) insertion of surfactin into the surface of a membrane by hydrophobic interaction between surfactin and the membrane; (2) generation of charge repulsions between negatively charged amino acids and negatively charged lipid headgroups of the surfactin structure which causes membrane bending; (3) membrane destabilization produces micelles which lead to membrane disintegration (Sebastien Buchoux et al., 2008; Huang et al., 2009). Studies reported by far suggest that the non-polar end of surfactin may interact with enveloped viruses and generates pores in the membrane which leads to formation of an ion channel and then destroys the entire envelope of these viruses (Vollenbroich et al., 1997). On the other hand, only limited studies relating to the capsid of non-enveloped viruses are available by far. However, Vollenbroich et al. believes that surfactin mainly causes damage to viruses by the interactions between its peptide moiety, not the carbon chain of fatty acids, and viral capsid (Vollenbroich et al., 1997). The difference between these two theories is one favors generation of leaks while the other proposes formation of pores. In summary, surfactin can cause damage to the membrane which further affects normal physiological function of cells. Studies have reported that antimicrobial peptides have the capacity to destroy Gram-negative bacteria, fungi and protozoa (Powers and Hancock, 2003). Furthermore, other literature also indicates that binding of AMP to the nucleic acids of prokaryotes inhibits protein synthesis (Powers and Hancock, 2003). Compared with conventional antibiotics, the activity of AMP has several features, such as broad-spectrum, high efficiency, stability, rapid bactericidal activity, and capability of interacting with the immune system.

Previous reports have indicated use of antibiotics as additives significantly destroys the intestinal microbial balance of animals, and its residues easily remain in the body which has great impact on the quality of livestock and human health. On the other hand, use of AMPs as feed additives can overcome the abovementioned problems and is the most direct application of AMP in animal husbandry. The AMPs in the intestinal tract of higher animals can inhibit exogenous pathogens while have no killing effects on normal intestinal flora and cells. The ingredients of AMPs are amino acids which can be easily digested and absorbed and thus AMPs can be used as feed additives to replace or partially replace antibiotics currently used in animals, and help to reduce the harm of antibiotics to animals. AMPs can replace traditional antibiotics to treat various problematic diseases such as diarrhea in pigs, swine fever, Newcastle disease and cows mastitis. In addition, AMPs will not generate drug-resistant bacteria, are non-toxic and produce no residues and thus have great application potential in treatment and prevention of animal diseases. Using AMPs as feed additives presents numerous advantages which have started to attract people's attention. It is very hopeful that AMPS will become one of the products to replace antibiotics. At present, silkworm liquid yeast preparation AD-peptide is the major product produced domestically and internationally for use as feed additive and most studies in livestock are also performed by using this AMP.

Mold contamination of feed and feed materials is a global problem. To reduce the occurrence of mold contamination, adopting proper measures to minimize the harm of mycotoxins to animal health has become the most important task for international animal husbandry, veterinary medicine, and feed technology sector over the years. Initially, people tried to control the water content of the feeds, environment temperature, and humidity to reduce the amount of mildew. With the advancement in chemistry and microbiology, chemical preservatives have been developed and the most common ones are organic acids, organic acid salts (esters) and composite mildew-proof agents, but they are all toxic after ingestion. Alternatively, AMP preservatives are proteins which can be easily degraded to amino acids in the GI tract, have only limited side effects in humans, and are edible. For example, Nisin has been wildly used in over 50 countries worldwide for food preservation. Non-toxic AMP preservatives made of natural ingredients have slowly replaced traditional chemical preservatives over the years and will play an important role in preservation of the products used in the feed industry and related fields in the future.

Due to increased use of antibiotics in livestock and aquatic animals, drug residues and generations of drug-resistant bacteria have worsened the safety issues of animal foods and posed a threat to human health as well as the development of aquaculture industry. Moreover, such problems have caused significant loss in export of livestock products. AMPs are considered to have great application potential because of their broad-spectrum antimicrobial activities, fast bacterial-killing effect, reduced possibility of generating drug-resistant bacteria, and synergistic effect with traditional antibiotics. Most importantly, AMPs demonstrate great diversity in their sequences and structures, which offers great possibility of imagination and development for designing new drugs and help to produce products with stable performance.

When used as feed additives, AMPs can withstand high temperature during the process of feed pelleting and such feature satisfies the requirements of the procedures for manufacturing feed additives. When used as antibiotics, AMPs exhibit unique mechanisms and have broad-spectrum antimicrobial activities, which helps to accelerate the growth of aquatic animals and livestock, maintain health, and treat diseases. In addition, AMPs have no toxic side effects, no residuals, and do not cause drug resistance. Also, the production cost is low and is not affected by environmental factors such as season or climate change. Industrial production of AMPs containing natural activity from aquatic microorganisms can meet the developmental needs of the aquatic and animal husbandry industry as well as help promote the development of green feed additives industry.

More studies have started to use AMPs as feed additives over the past few years and this field is still at the stage of exploration. Before mature technology can be developed and large-scale production can become possible, many problems need to be resolved. First, the natural resources of AMPs are limited and thus chemical synthesis and genetic engineering have become the major means to obtain AMPs. Second, the costs of synthesis is expensive and mass production is still not possible. Third, through genetic engineering, direct expression of the AMP genes in microorganisms may lead to death of the microorganisms or too little of product expression. Most production models employed at present for generation of AMPs mainly produce AMPs by liquid fermentation of engineered bacterial strains and have numerous disadvantages, including that the costs for production equipment and liquid fermentation invested at the early stage are very high while the return on investment is slow, and using the engineered bacterial stains as feed additives directly without purification may raise the doubt of GMO additives. Therefore, development of AMPs that are natural, have broad spectrum of antimicrobial or bacterial-killing activity, are cheap, and are non-GMO to replace antibiotics is an important task for medical/pharmaceutical field and development of feed additives.

The present invention uses a semi-solid state fermentation method to produce surfactin from a high-yield mutant strain of Bacillus subtilis by using soybeans as the fermentation substrate, and the resulting soybean powder is used as feed additive after drying and grinding the fermented soybeans.

### SUMMARY OF THE INVENTION

The present invention provides a method for preparation of feed additives containing surfactin.

This method comprises the following steps:

Step 1: Inoculation of the bacterial culture of the high-yield mutant strain Bacillus subtilis TH to a mixture containing 30% mineral salt medium and soybeans and incubate at 30-40° C., humidity 80-90% for semi-solid fermentation for 2-3 days to give fermented soybeans. The ratio of the volume of inoculated bacterial culture of the high-yield mutant strain of Bacillus subtilis to the volume of soybeans in the mixture is 5:100-10:100.

Step 2: Drying the fermented soybeans at 50-60° C. for 2-3 hours before grinding and sieving to give soybean powder rich in surfactin (6-7 g per kg). Said soybean powder is the feed additive containing surfactin and the amount of surfactin is 6-7 g per kg.

The present invention also provides a method for preparation of feed additives containing surfactin and said feed additive is used as the feed for aquatic animals such as grouper or white shrimp, has the activity to promote the growth rates of aquatic animals and enhance immunity, and has antimicrobial, anti-fungal and anti-viral activity.

The present invention also provides a feed for aquaculture, comprising the additive prepared according to the aforementioned method.

The present invention also provides a method for maintaining health of aquatics, comprising administering the effective dosage of the feed of claim 13 to the aquatics in need.

### BRIEF DESCRIPTION OF THE DRAWINGS

FIG. 1 shows neutralizing titration results of surfactin at different concentrations in virus-infected cell lines.

FIG. 2A shows the effect of surfactin on morphology of nervous necrosis virus particles: (A) 1% L-15 treatment group; (B) 1% L-15+100 μg/ml surfactin mixed treatment; (C) 1% L-15 nervous necrosis virus-mixed treatment group; (D) 40 μg/ml surfactin+nervous necrosis virus mixed treatment group, the arrow indicates normal viral particles; (E) 100 μg/ml surfactin and nervous necrosis virus mixed treatment group and the arrow indicates abnormal viral particles.

FIG. 2B shows the effect of surfactin on morphology of Iridovirus particles: (A) normal Iridovirus group; (B) 10 μg/ml surfactin+Iridovirus treatment group, the appearance of said virus has changed; (C) 40 μg/ml surfactin+Iridovirus mixed treatment group, adhesion between surfactin and viruses is observed.

FIG. 3 shows promotion of white shrimp growth by the feed additive containing surfactin.

FIG. 4 shows promotion of grouper growth by feed additive containing surfactin.

FIGS. 5A, 5B, and 5C show the expression of immune genes is increased by the feed additive containing surfactin.

FIG. 6A shows the food additive containing surfactin reduces the mortality of grouper infected with nervous necrosis virus.

FIG. 6B shows the food additive containing surfactin reduces the mortality rate of grouper infected with Iridovirus.

### DETAILED DESCRIPTION OF THE PREFERRED EMBODIMENT

### Example 1

### Preparation of the Food Additive Containing Surfactin by Semi-Solid State Fermentation

This patent utilizes high-yield mutant strain of Bacillus subtilis (BCRC No. CGMCC 10270) for semi-solid state fermentation by using soybeans as the substrates. The surface of the fermented soybeans is rich in secondary metabolite-surfactin. The substrate of fermentation can be other cheaper substrates such as rape seed meal, palm oil cake, coconut oil cake, poonac, agricultural by-products or waste and trash (potato peel, cassava peel and apple peel).

The bacterial culture of high-yield mutant strain of Bacillus subtilis was inoculated to the mixture of 30% mineral salt medium and 10% soybeans and subjected to semi-solid state fermentation for 48 hours under the condition of 30° C., 80% humidity, followed by drying the fermented soybeans at 55° C. for 2-3 hours. After grinding, the soybeans were sieved by a 60-mesh sieve and stored at 4° C., wherein each kilogram of the soybean powder contains 6-7 g of surfactin.

### Example 2

### Antimicrobial Testing for Surfactin

To examine whether surfactin has antimicrobial activity, in vitro susceptibility test was conducted.

Preparation of bacterial culture of Escherichia coli DH5α, Vibrio harveyi, Vibrio alginolyticus, Vibrio anguillarum, Vibrio salmonicida, Aeromonas hydrophila and Staphylococcus epidermidis by inoculation of these bacteria onto LBA (E. coli·DH5α) or TSA (+1.5% NaCl) and incubation at 37° C. for 16 hours. Later, the colonies were scraped off and dissolved in specific medium; the concentration was adjusted to OD₅₄₀ of 1 (1×10⁹ colonies/c.c. culture medium), 100 μl of bacterial culture were add to 900 μl of LB or TSB (+1.5% NaCl) to give a concentration of 5×10⁵ CFU/ml.

Preparation of Fungal Cultures

Aspergillus niger was inoculated onto the MEAIII plate and incubated at 25° C. for 48 hours, the colonies were scraped off into specific culture medium. Bacterial culture was spread onto the MEAIII plate after serial dilution and the number of colonies was calculated, the original concentration was determined to be 1×10⁶ CFU/ml. An aliquot of 500 μl of the bacterial culture was add to 500 μl of MEB before diluting the concentration of bacterial culture to 5×10⁵ CFU/ml.

Susceptibility Test

An aliquot of 130 μl of the bacterial culture was added to each well of a 96-well plate, the concentration of bacterial culture was 5×10⁵ CFU/ml, 20 μl of surfactin at different concentrations prepared by titration were added and incubated at 37° C. for 16 hours. A spectrophotometer was used to calculate the concentration of bacterial growth and the obtained data was compared with the original value (OD₅₄₀=1), the concentration of the experimental group that equals to the original value is used as the minimum inhibiting concentration (MIC). Each group was prepared in triplicate and a control group was included.

The results are shown in Table 1. When compared with the surfactin produced by Bacillus subtilis ATCC21332, the MIC of the surfactin produced by the high-yield mutant strain of Bacillus subtilis was 96.5 μM.

TABLE 1
Comparison of the minimum inhibiting
concentration (MIC) (μM) of surfactin produced
by Bacillus subtilis ATCC21332 and the
high-yield mutant strain of Bacillus subtilis.

| | Bacillus | High-yield |
|---|---|---|
| | subtilis | mutant |
| | ATCC21332 | strain |
| Gram positive | | |
| E. coli | 193 | 96.5 |
| Aeromonas | 128.7 | 96.5 |
| Vibrio anguillarum | 128.7 | 96.5 |
| Vibrio alginolyticus | 128.7 | 96.5 |
| Vibrio harveyi | 128.7 | 96.5 |
| Vibrio salmonicida | 128.7 | 96.5 |
| Gram negative | | |
| Staphylococcus | 128.7 | 96.5 |
| epidermidis | | |
| Fungi | | |
| Aspergillus niger | 193 | 128.7 |

### Example 3

### Neutralization Test of Surfactin and Nervous Necrosis Virus (NNV)

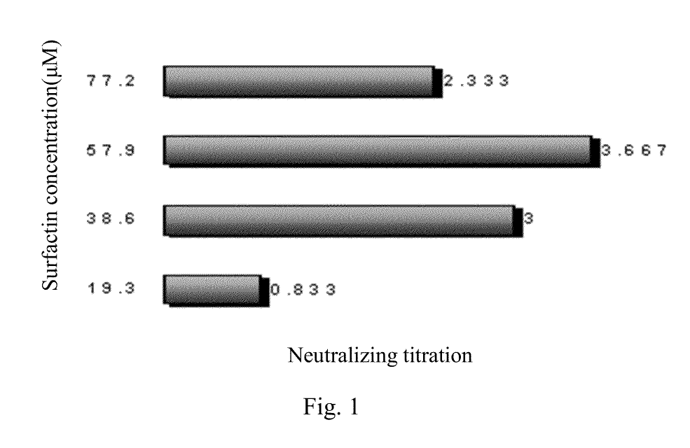

To examine whether surfactin has anti-viral activity, neutralization test was conducted. One day before the test, GF-1 cells were seeded onto a 96-well microplate at the density of 5×10³ cells per well and cultured overnight (16 hours) until 80% confluency before subjected to titration. First, a 10-fold serial dilution, from 10¹ to 10¹⁰, of the test viral culture was performed and then the diluted viral culture was inoculated onto a 96-well plate, 100 μl per well. Shake the plate to mix the viral culture, a total of 8 repeats and incubate the plate at 28° C. for 90 minutes to allow virus adsorption. The old culture medium was replaced with 200 μl of 1% FBS-L15 culture medium and the plate was incubated the 28° C. for additional 9 days. The 50% tissue culture infection dose (TCID50) was calculated based on the Reed-Muench Method and is represented by TCID50 for neutralization index (NI=original virus titer/NI titer after surfactin neutralization), the higher the NI titer, the higher the neutralization titer of surfactin. The neutralization reaction was conducted as a 10-fold serial dilution. The neutralization potency is determined as having no neutralization potency when the NI is less than 1 (i.e. the value of Log₁₀NI is less than 1). A NI ranging from 10 to 50 (the value of Log₁₀NI is between 1 and 1.6) indicates the neutralization potency is not significant and is in a doubtful range. A NI over 50 (the value of Log₁₀NI is greater than 1.7) is determined as having significant neutralization potency (Grace, 1979). The results are shown in FIG. 1.

### Example 4

### The Mechanism of the Interactions of Surfactin with Virus

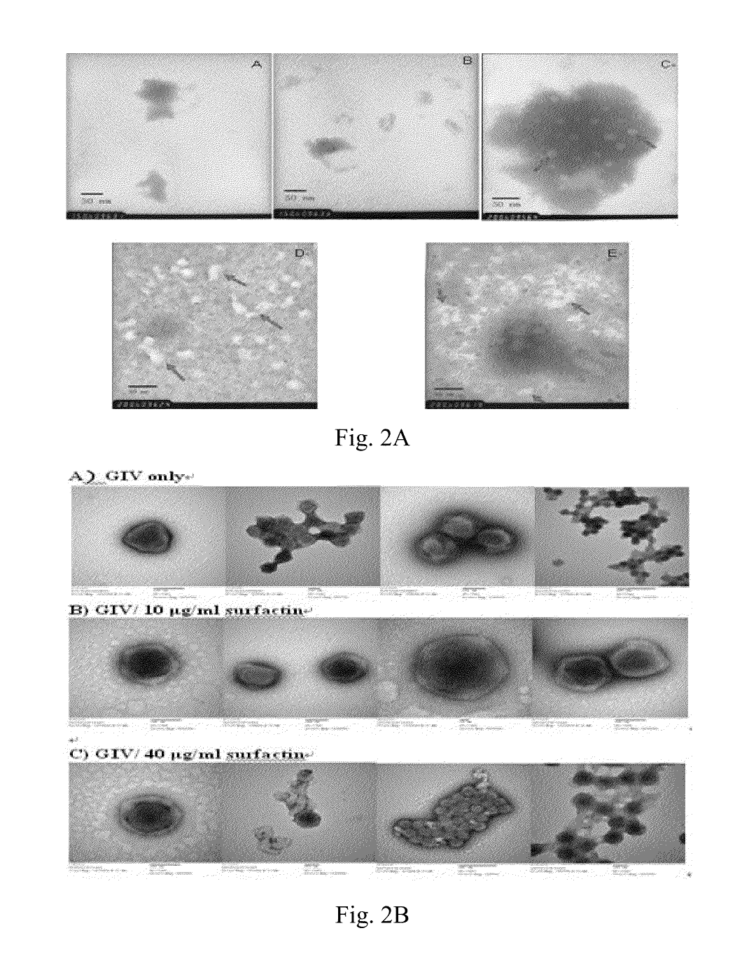

Surfactin was dissolved in sterilized water and the solution was filtered with a 0.2 μm filter membrane, followed by dilution of dissolved surfactin in 1% FBS-L15 to give diluted surfactin solution at the concentrations of 40 μg/ml and 100 m/ml. The surfactin treatment group: surfactin and virus in the volume ratio of 1:1 were mixed. Control group: 1% FBS-L15 culture medium with NNV culture were mixed and incubated at 28° C. for 1 day before examination under a transmission electron microscope (Hitachi H-600). An aliquot of 6 μl of the surfactin treatment group was dropped onto a copper mesh to allow precipitation of the virus for about 10 minutes before been stained in 1% phosphotungstic acid (PTA, Sigma) at pH 7.4. After staining, excess mixture was removed with filter paper and incubated at room temperature for 30 minutes. The sample was examined after the copper mesh was air-dried. The voltage used for transmission electron microscope was 75 KV and the exposure time of the film was 4 seconds. The removed negative film was placed on the rack and soaked in the developer D19 for 4 minutes to develop the image. After completion of development, the film was soaked in 0.33% glacial acetic acid to stop the reaction followed by 10 minutes in fixative. Finally, excess chemicals were removed by placing the film under running water for 60 minutes before drying. The results are shown in FIG. 2.

### Example 5

### Growth Test of Increasing the Growth Rate of White Shrimp by Feed Additives Containing Surfactin

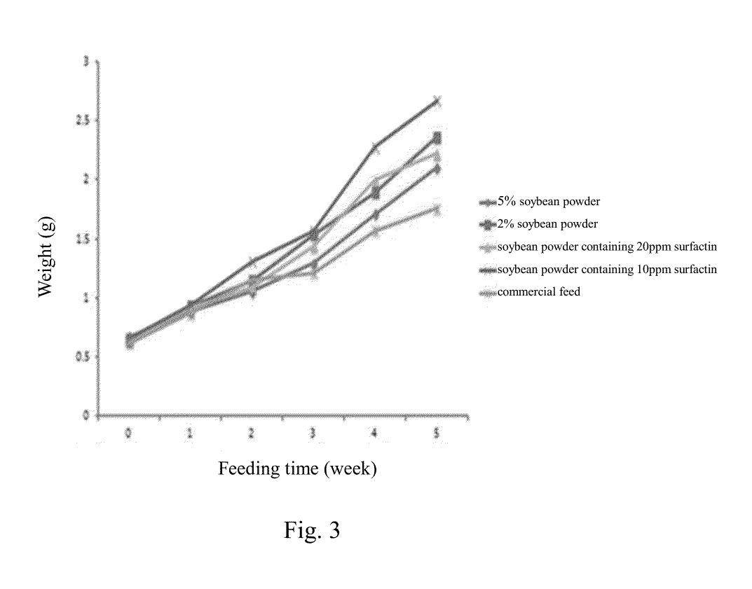

The fermented soybeans and commercial shrimp feed were grounded and then pelletized by using a pellet feed mill after addition of sterilized water, followed by drying at 70° C. in an oven for 3 hours to reduce the water content in the feed to 10% and the dried feed was stored at 4° C. The growth test includes 4 feed treatment groups: 1. addition of fermented soybean powder which contains 10 ppm of surfactin, 2. addition of fermented soybean powder which contains 20 ppm of surfactin, 3. addition of water-washed fermented soybean powder 20%, 4. addition of water-washed fermented soybean powder 5%, and control group: commercial feed. Each group includes 25 white shrimps fry and the wet weights were around 0.6 g. The experimental tank was a 30 cm×24 cm×60 cm transparent glass cube containing 45 liters of water without temperature control. An air pump was turned on during the entire period of the experiment, and about ⅓ of water was changed each time. The daily feeding amount was 5% of the weights of shrimps and the feeding times were 8 A.M. and 5 P.M. every day. The wet weights were measured and recorded every week for calculation of the growth rate. The results are shown in FIG. 3.

### Example 6

### Growth Test of Increasing the Growth Rate of Grouper by Feed Additive Containing Surfactin

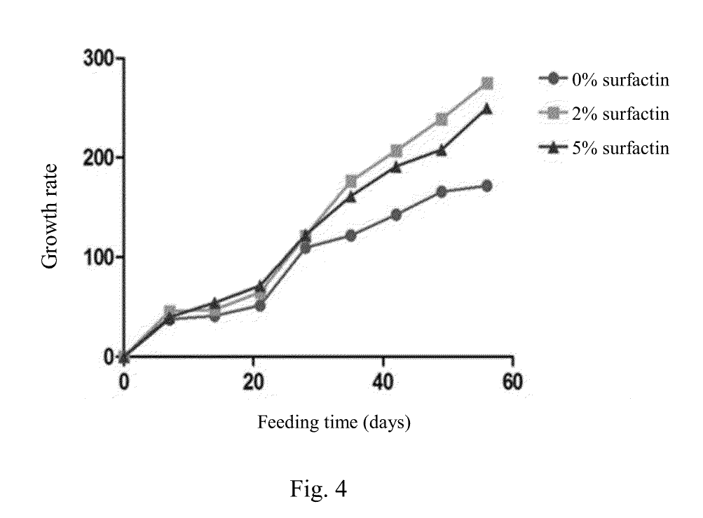

The protein and fat content of the feed were formulated based on the study of Shiau and Lan (1996). The formula of the test feed was common feed with 47% crude protein and 10% fat. Red fish powder, corn gluten protein, wheat gluten protein and squid powder were ground through a grinder and sieved (35 mesh screen) to increase viscosity of the feed. All feeds were pelletized by a pellet feed mill before separation and drying and 2-5% of surfactin was then added before storing at −20° C. to ensure feed quality. The results are shown in FIG. 4.

### Example 7

### Feed Additives Containing Surfactin can Induce Innate Immune Gene Expression in the Animals

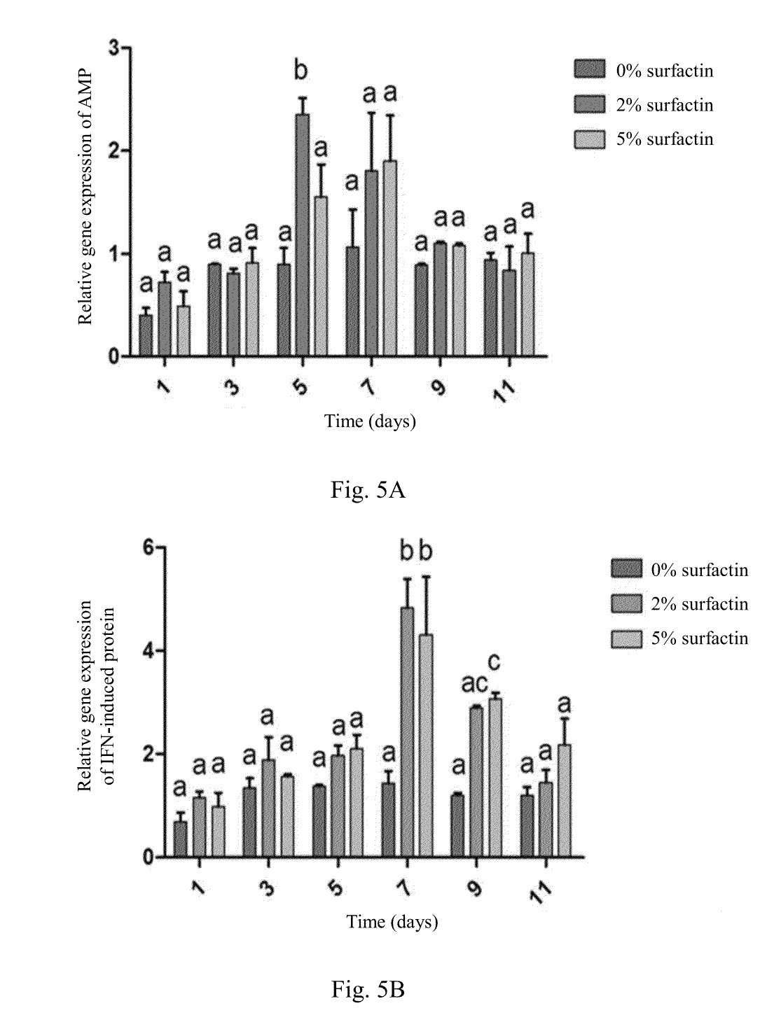

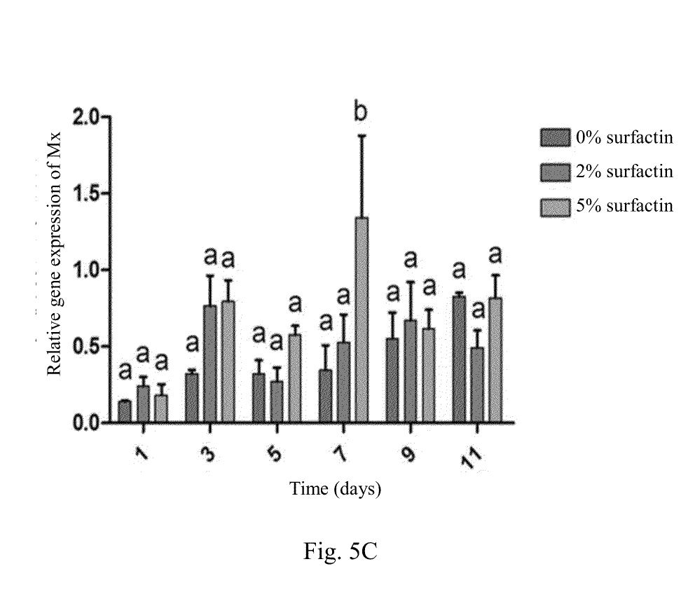

Continuous feeding of grouper with feeds containing 0%, 2% and 5% surfactin can induce expression of the immune genes in grouper including AMP (Antimicrobial peptide), MX (MX protein is a GTPase induced by interferon) and INF-inducted protein. The results are shown in FIG. 5.

### Example 8

### Feed Additive Containing Surfactin Increases Disease Resistance of Animals

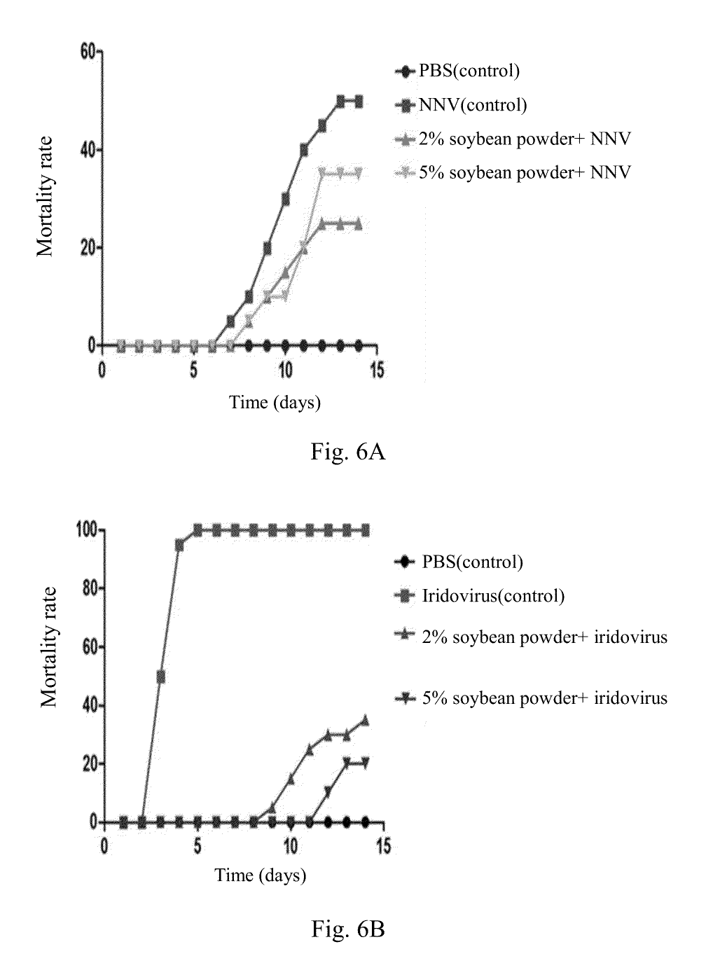

Change in mortality rate in groupers infected with either NNV or Iridovirus after giving the feed containing surfactin for 7 consecutive days to orange spotted grouper fry. The experiment was divided into 4 groups: 1. negative control group: feed with common feed+PBS (phosphate-buffered saline) injection; 2. positive control group: fee with common feed+virus injection; 3. study group: feed with the feed containing 2% surfactin+virus injection; 4. study group: feed with the feed containing 5% surfactin+virus injection. The results are shown in FIG. 6.

The surfactin produced by the method of present invention disclosed in the abovementioned embodiments can be used in aquaculture feeds and be adapted to the changes of water environment and numerous pathogens; in addition, said surfactin is acid(base)-resistant, heat-resistant, has broad-spectrum antimicrobial activity (Gram-positive and negative, fungi, parasites, viruses), does not cause drug resistance and can be bio-degraded. Said surfactin is environmental-friendly and a green feed additive without the problem of drug residues. It is very suitable for use as a new kind of antibiotic and anti-disease feed additive.

The method of the invention uses semi-solid state fermentation and dries and grinds the substrate directly after fermentation to give the feed additive which can replace common antibiotics. The production process of this method is simple and the production cost is cheap.

1. A method for preparation of feed additives containing surfactin, comprising the following steps:
inoculating the bacterial culture of the high-yield mutant strain Bacillus subtilis TH to a mixture containing 30% mineral salt medium and soybeans for semi-solid fermentation to produce fermented soybeans;
drying the fermented soybeans before grinding and sieving to produce a soybean powder, said soybean powder being the feed additive containing surfactin.

2. The method of claim 1, wherein the BCRC number of the high-yield mutant strain is CGMCC 10270.

3. The method of claim 1, wherein the volume ratio of inoculated bacterial culture of the high-yield mutant strain of Bacillus subtilis to soybeans in the mixture is 5:100-10:100.

4. The method of claim 1, wherein the semi-solid state fermentation step comprises incubating for 2-3 days at 30-40° C. and 80-90% humidity.

5. The method of claim 1, wherein the fermented soybeans are dried at a temperature 50-60° C. for 2-3 hours.

6. The method of claim 1, wherein the soybean powder contains 6-7 g/kg of surfactin.

7. The method of claim 1, wherein the feed additive is used in aquaculture feeds.

8. The method of claim 7, wherein the aquaculture is fish or shrimp.

9. The method of claim 8, wherein the fish is grouper and the shrimp is white shrimp.

10. The method of claim 1, wherein the feed additive is used to promote the growth rate of aquatic organisms.

11. The method of claim 1, wherein the feed additive is used to increase the immunity of aquatic organisms.

12. The method of claim 1, wherein the feed additive has anti-bacterial, anti-fungal and anti-viral activity.

13. A feed for aquaculture, comprising the additive prepared according to the method of claim 1.

14. A method for maintaining health of aquatics, comprising administering an effective dosage of the feed of claim 13 to an aquatic subject in need thereof.
